(19) United States
(12) Patent Application Publication
(10) Pub. No.: US 2026/0232858 A1
(43) Pub. Date: Aug. 13, 2026

(54) Device for Holding a Medical Instrument in a Disinfection Chamber and Associated Assembly

(71) Applicant: GERMITEC, Bordeaux (FR)

(72) Inventors: Fanny POULON, Bures Sur Yvette (FR); Maxime BERTHIAS, Cadarsac (FR); Coralie DUMAS, Montreal (FR)

(21) Appl. No.: 19/535,697

(22) Filed: Feb. 10, 2026

(30) Foreign Application Priority Data
Feb. 11, 2025 (FR) FR 25 01382

(51) Int. Cl. A61L 2/26; A61L 103/15

(52) U.S. Cl. CPC A61L 2/26; A61L 2103/15

(57) ABSTRACT

This invention relates to a device for holding (14) for a medical instrument in a disinfection chamber, the device (14) comprising:

- two half-shells (34, 35) able to cooperate in a holding position; and

- a locking device (36) able to maintain the two half-shells (34, 35) in the holding position around a part of the medical instrument, the locking device (36) comprising two complementary locking elements (54).

One of the locking elements comprises a first component of a first magnet, the first component being of a first polarity, the other of the locking elements comprising a second component of the first magnet, the second component being of a second polarity.

### DESCRIPTION

This invention relates to a device for holding a medical instrument in a disinfection system.

In order to avoid any contamination, it is necessary to disinfect a medical instrument between two uses.

To this end, it is known to disinfect medical instruments in disinfection chambers defining a closed disinfection volume. Such chambers comprise, for example, a device for generating UV (Ultra-violet) radiation, allowing notably to carry out disinfection of these medical instruments, and more particularly a high-level disinfection.

Medical instruments, such as medical probes, for example, typically comprise at least one active part and generally, also at least one connecting part in the form of a cable.

It is known to suspend these medical instruments in the disinfection chamber, for example, using a ring attached to a portion of the connecting part and adapted to cooperate with a support of the disinfection chamber.

Such a ring is, for example, constituted of half-shells connected by screws.

However, such a ring is not entirely satisfactory, particularly due to the presence of screw holes that can lead to a lack of sealing. Such a lack of sealing can result in insufficient hygiene of the ring, and for example, contamination by embedding impurities in the holes.

In addition, such a ring is generally compatible with a very limited number of connecting cable diameters.

Furthermore, such a ring generally has a very limited lifespan, notably less than 10 uses, in particular due to the limited lifespan of the screw threads.

In addition, it is known to identify the medical instrument in the disinfection chamber, in order to ensure traceability of the disinfection. For this purpose, one of the half-shells of the ring comprises, for example, a window behind which an identification label, for example with a barcode, is held.

However, such a window creates additional gaps, which increases the risk of insufficient sealing.

One of the aims of the invention is to overcome these drawbacks, and notably to propose a sealed, durable, and ergonomic device allowing to hold various medical instruments in a disinfection chamber.

To this end, the invention has as its object a device for holding a medical instrument in a disinfection chamber, the device comprising:

two half-shells able to cooperate in a holding position to form together a holding element, each half-shell comprising a casing extending according to a casing axis and defining an external face and an internal face; and

a locking device able to maintain the two half-shells in the holding position around a part of the medical instrument, the locking device comprising two complementary locking elements able to cooperate with each other, one of the locking elements being arranged on one of the half-shells and the other locking element being arranged on the other half-shell.

One of the locking elements comprises a first component of a first magnet, the first component being of a first polarity.

The other of the locking elements comprises a second component of the first magnet, the second component being of a second polarity opposite to the first polarity.

The use of a locking device with a magnet allows to avoid the need to define additional gaps, such as screw holes, for example, which increases the sealing of the device.

Furthermore, locking by cooperation of magnetic components is ergonomic and durable.

According to other advantageous aspects of the invention, the device comprises one or more of the following features, taken alone or in any technically possible combination:

The internal face of each half-shell defines a housing, in the holding position, the two housings together forming a receiving space for the part of the medical instrument.

The device further comprises a seal configured to be arranged between the part of the medical instrument and the holding element.

The seal is configured to extend throughout the entire receiving space formed by the two housings in the holding position.

One of the locking elements also comprises a first component of a second magnet, the first component being of a first polarity, the other of the locking elements also comprising a second component of the second magnet, the second component being of a second polarity.

The first magnet and/or the second magnet are neodymium super-magnets.

The internal face of each half-shell defines at least one cavity filled with a filling agent.

Each element of the first magnet, and if applicable each element of the second magnet, is arranged in the or one of the cavities of the corresponding half-shell.

The device comprises an identification element engraved on the external face of one of the half-shells.

The invention also relates to an assembly comprising:

a device such as described above, and

a medical instrument comprising a part able to be held in the holding element formed by the two half-shells.

### BRIEF DESCRIPTION OF THE DRAWINGS

The invention will become clearer upon reading the following description, given solely by way of non-limiting example, and made with reference to the drawings wherein:

FIG. 1 is a schematic representation of an assembly according to the invention in a disinfection chamber, the assembly comprising a device for holding,

FIG. 2 is an exploded schematic representation of the device for holding of FIG. 1, and

FIG. 3 is a schematic representation of one of the half-shells of the device for holding of FIG. 1.

### DETAILED DESCRIPTION

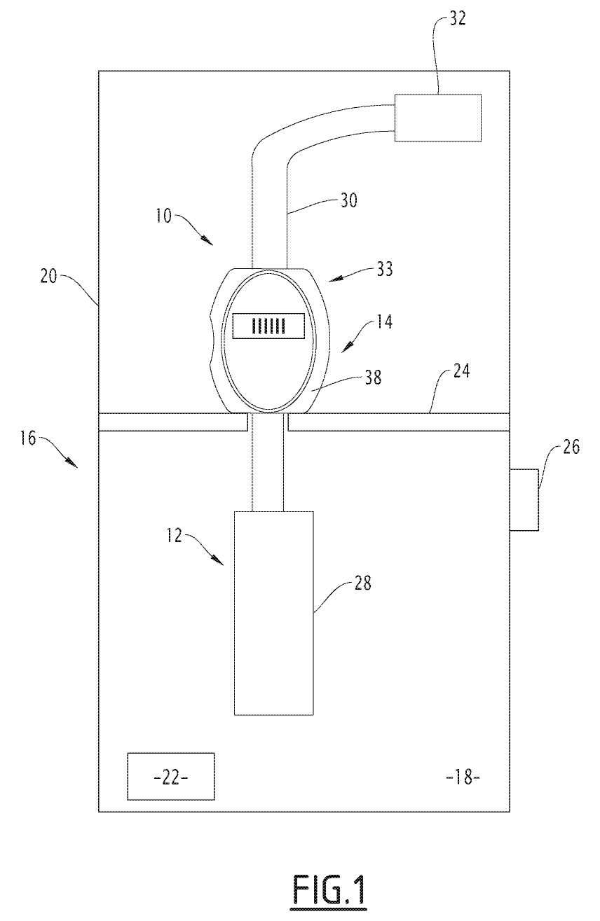

FIG. 1 shows an assembly 10 comprising a medical instrument 12 and a device 14 for holding in a disinfection chamber 16.

In particular, in FIG. 1, the medical instrument 12 is held in the disinfection chamber 16 by the holding device 14.

For example, the disinfection chamber 16 comprises a disinfection volume 18 delimited by at least one wall 20, a disinfection unit 22, and at least one support 24.

Preferably, the disinfection chamber 16 also comprises an identification unit 26.

The disinfection unit 22 is, for example, a device for radiation generation, generating UV radiation, preferably UV-C.

More specifically, the disinfection unit 22 is, for example, configured to generate UV radiation, and notably UV-C, in the disinfection volume 18.

The support 24 is preferably configured to cooperate with the holding device 14 so as to hold the medical instrument 12 inside the disinfection volume 18.

The identification unit 26 is, for example, configured to identify the medical instrument 12.

By "identify," it is meant more particularly the reading of identification information of the medical instrument 12.

In a particular embodiment, the identification unit 26 is a barcode reader, for example arranged on an external face of one of the walls of the disinfection chamber 16.

The medical instrument 12 is, for example, a medical probe, notably an ultrasound medical probe, and more particularly an endocavity ultrasound medical probe, such as, for example, a gynecological probe.

The medical instrument 12 comprises, for example, an active part 28. The active part 28 is, for example, a tube intended to be inserted into the patient.

The medical instrument 12 also preferably comprises a connecting part 30 and in an even more preferential manner a connector 32.

The connecting part 30 is, for example, a connecting cable linking the active part 28 to the connector 32, as is notably visible in FIG. 1.

More particularly, the connecting part 30 is, for example, a cable with a diameter between 3mm and 10mm.

The connector 32 is, for example, configured to be connected to a measurement and/or display unit, notably so as to collect and/or display information captured by the active part 28.

The holding device 14 comprises two half-shells 34, 35, and a locking device 36.

As visible in FIG. 1, the two half-shells 34, 35 are able to cooperate in a holding position, in particular to form together a holding element 33 (visible in FIG. 1), for example around a part of the medical instrument 12, and more particularly around the connecting part 30 of the medical instrument 12.

The holding element 33 thus formed has, for example, a cross-section of substantially circular or oval shape.

The locking device 36 is able to maintain the two half-shells 34, 35 in the holding position around a part of the medical instrument 12, and in particular, to maintain the holding element 33 around the part of the medical instrument 12.

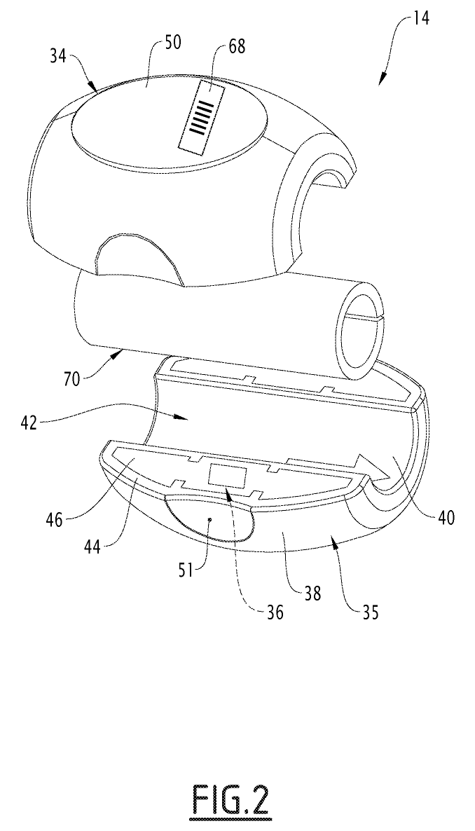

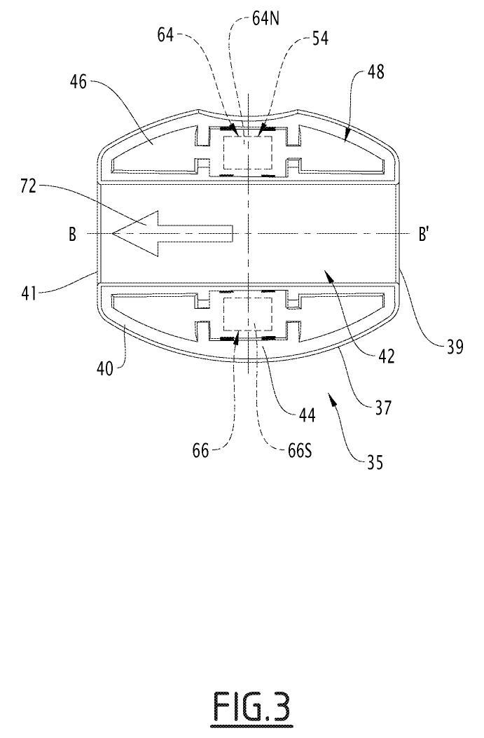

FIG. 3 shows one of the half-shells 35 of the device for holding 14 of FIG. 2.

The two half-shells 34, 35 of the device for holding 14 are preferably substantially identical.

Each half-shell 34, 35 comprises a casing 37 extending according to a casing axis B-B'.

Each casing 37 is, for example, made of plastic.

When the half-shells 34, 35 cooperate together to form the holding element 33 around a part of the medical instrument 12, the casing axis B-B' is preferably aligned with the axis of the connecting part 30 of the medical instrument 12.

Each half-shell 34, 35 defines a first end 39 and a second end 41 opposite the first end 39 according to the casing axis B-B'.

As illustrated in FIG. 1, the first end 39 and the second end 41 correspond, for example, to a top end and a bottom end of the casing 37, when the half-shells 34, 35 cooperate to form the holding element 33 around the vertically extending connecting part 30 of the medical instrument 12.

Each half-shell 34, 35 defines an external face 38 (visible in FIGS. 1 and 2) directed radially outward and an internal face 40 (visible in FIGS. 2 and 3) directed radially inward, and more particularly intended to be directed toward the connecting part 30 of the medical instrument 12.

More precisely, a part of the internal face 40 is intended to come against the medical instrument 12, and in particular against the connecting part 30 of the medical instrument 12, or to come against the seal 70 (which will be described later).

The internal faces 40 of each half-shell 34, 35 are advantageously able to come partially into contact with each other when forming the holding element 33.

In particular, each of the internal faces 40 comprises a part intended to come against the medical instrument 12 or against the seal 70 and advantageously at least another part intended to come against the internal face 40 of the other half-shell 34, 35.

Preferably, the internal face 40 of each half-shell 34, 35 defines a housing 42.

As illustrated in FIGS. 2 and 3, the housing 42 preferably extends according to the casing axis B-B' from the first end 39 to the second end 41.

The housing 42 has, for example, a length, notably corresponding to the distance between the first end 39 and the second end 41, between 30mm and 60mm.

For example, the housing 42 presents a section, of semi-circular form, with a diameter slightly larger than that of the connecting part 30 of the medical instrument 12, notably between 10mm and 15mm.

Preferably, in the holding position, the two housings 42 together form a receiving space for the part of the medical instrument 12, the receiving space being, for example, cylindrical.

As visible in FIGS. 2 and 3, the internal face 40 of each half-shell 34, 35 advantageously defines two edges 44 extending on each side of the housing 42.

Preferably, the edges 44 of the internal faces 40 of each half-shell 34, 35 come into contact with each other when forming the holding element 33.

Advantageously, as visible in FIGS. 2 and 3, the internal face 40 of each half-shell 34, 35 defines at least one cavity 46, and preferably two cavities 46.

In a preferred manner, each cavity 46 is arranged in one of the edges 44 of the internal face 40 of the casing 37.

Advantageously, each cavity 46 is filled with a filling agent 48, and preferably completely filled.

By completely filled, it is meant that the filling agent 48 is present in the total volume of the cavity 46.

In particular, the cavity 46 is filled so that the filling agent 48 is flush with the top of the edge 44.

The filling agent 48 is, for example, resin, notably epoxy resin.

The external face 38 of each half-shell 34, 35 comprises, for example, a flat surface 50, for example of circular or oval shape.

Preferably, the external face 38 of each half-shell 34, 35 comprises a recess 51, preferably of semi-circular shape.

Advantageously, in the holding position, the two recesses 51 together form a notch of substantially circular shape, adapted for the positioning of a thumb of the user.

The locking device 36 comprises two complementary locking elements 54 able to cooperate with each other, one of the locking elements being arranged on one of the half-shells 34 and the other locking element 54 being arranged on the other half-shell 35.

By cooperation of the locking elements 54 of the locking device 36, the holding element 33 formed by the two half-shells 34, 35 is able to be maintained on the part of the medical instrument 12 and more particularly as explained previously according to the radial direction.

One of the locking elements comprises a first component of a first magnet 64, the first component being of a first polarity, corresponding, for example, to the south magnetic pole.

The other locking element 54 comprises a second component 64N of the first magnet 64, the second component 64N being of a second polarity opposite to the first polarity, corresponding, for example, to the north magnetic pole.

In one preferred embodiment visible in FIGS. 1 and 2, one of the locking elements 54 also comprises a first component of a second magnet 66, the first component 66S being of a first polarity, corresponding, for example, to the south magnetic pole. The other locking element also comprises a second component of the second magnet 66, the second component being of a second polarity, corresponding, for example, to the north magnetic pole.

Preferably, the first magnet 64 and/or the second magnet 66 are neodymium super-magnets.

Each of the first and second components 64N of the first magnet 64, and respectively each of the first component 66S and the second component of the second magnet 66, if applicable, are more particularly arranged facing each other in the holding position, notably to create a magnetic attraction force between them fixing the two half-shells 34, 35 together.

Each of the first and second components 64N of the first magnet 64, and respectively each of the first and second components 66S of the second magnet 66, if applicable, is, for example, arranged on one of the edges 44 of the internal face 40 of the corresponding half-shell 34, 35.

More precisely, each component 64N of the first magnet 64, and preferably each component 66S of the second magnet 66, is arranged in one of the cavities 46 arranged in the internal face 40 of the corresponding casing 37.

Preferably, each component 64N of the first magnet 64, and preferably each component 66S of the second magnet 66, is completely embedded in the filling agent 48 in the corresponding cavity 46.

By completely embedded, it is meant that the filling agent 48 completely covers the component in the cavity 46.

In a preferential manner, the device for holding 14 also comprises an identification element 68.

In particular, the identification element 68 comprises identification information, comprising, for example, an identification number of the device for holding 14 and/or the medical instrument 12 intended to be disinfected.

The identification element 68 is, for example, a barcode.

Preferably, the identification element 68 is engraved on the external face 38 of one of the half-shells 34, 35, and for example by laser engraving.

For example, as visible in FIGS. 1 and 2, the identification element 68 is engraved on the flat surface 50 of one of the half-shells 34, 35.

In a preferential manner, the device for holding 14 also comprises a seal 70 configured to be arranged between the part of the medical instrument 12 and the holding element 33.

In particular, the seal 70 is configured to enclose the portion of the connecting part 30 intended to be received in the receiving space formed by the two housings 42 in the holding position.

Advantageously, the seal 70 is configured to extend throughout the entire receiving space formed by the two housings 42 in the holding position, which notably allows to reduce the risk of liquid penetration into the holding element.

Preferably, the seal 70 has a length substantially identical to that of the housings 42.

For example, as visible in FIG. 2, the seal 70 is a split cylinder, in particular able to be inserted around the connecting part 30 of the medical instrument 12.

In an advantageous manner, the seal 70 presents an annular section with an internal diameter substantially equal to that of the connecting part 30 and an external diameter substantially equal to that of the housings 42.

The seal 70 is, for example, made of a material comprising silicone, and in particular silicone rubber.

In an advantageous manner, the device for holding 14 also comprises at least one pictogram 72, for example in the shape of an arrow as visible in FIG. 2.

This pictogram 72 is preferably engraved, for example by laser engraving, on one of the faces 38, 40 of one of the half-shells 34, 35.

For example, the pictogram 72 is engraved on the internal face 40 of one of the half-shells 34, 35, and in particular in the corresponding housing 42, so as to indicate to the user a direction of insertion of the connecting part 30.

An example of use of such a device for holding 14 will now be described.

A device for holding 14 such as described above and a medical instrument 12 intended to be disinfected are first provided.

In a preferential manner, the seal 70 is fixed on the medical instrument 12.

More particularly, the seal 70 is fixed around a portion of the connecting part 30 of the medical instrument 12.

A part of the medical instrument 12, in particular the assembly, formed by the seal 70 and the portion of the connecting part 30 surrounded by it, is positioned in the housing 42 of one of the half-shells 34, 35.

The other half-shell 35 is then fixed to it in the holding position by the locking device 36, so as to form the holding element 33.

For example, the device for holding 14 is identified, notably by reading the information on the identification element 68 by the identification unit 26 of the disinfection chamber 16.

The assembly formed by the device for holding 14 and the medical instrument 12 is then arranged in the disinfection chamber 16, notably by cooperation between the holding element 33 and the support 24 as visible in FIG. 1, so as to suspend the medical instrument 12.

A disinfection cycle of the medical instrument 12 is then, for example, implemented, notably by the disinfection unit 22.

Such a device for holding 14 is particularly advantageous.

In particular, the use of a locking device 36 with at least one magnet 64, 66 allows to avoid the need to define additional gaps, such as screw holes, which increases the sealing of the device.

Similarly, the removal of the window for identification by engraving the identification element 68 also enhances this sealing.

Furthermore, the design of the half-shells 34, 35 without hollows, notably by embedding the components 64N, 66S of the magnets 64, 66 in the filling agent 48, further strengthens this sealing.

Such a device for holding 14 therefore presents optimized sealing, which reduces the risk of contamination.

Furthermore, the absence of gaps and hollows facilitates the cleaning of the device for holding 14.

In addition, locking by cooperation of magnet components 64N, 66S is simple to implement and ergonomic.

The optional addition of recesses 51 on the half-shells 34, 35 further facilitates the handling of such a device for holding 14 and makes it even more ergonomic.

Furthermore, by eliminating the screw thread thanks to the use of magnets 64, 66, the durability of such a device for holding is greatly increased.

In addition, the engraving of the identification element 68 enhances its physical integrity, thus preventing it from becoming unreadable, which limits the risk of misidentification.

The use of the seal 70 also allows to make this device for holding 14 compatible with a large number of connecting part 30 diameters, notably by choosing an appropriate thickness of the seal 70.

In addition, such a device for holding 14 is simple to manufacture and economical, notably due to its low number of components.

The skilled person will understand that the embodiments and alternatives previously described can be combined with each other provided they are technically compatible.

1. A device for holding a medical instrument in a disinfection chamber, the device comprising:
two half-shells able to cooperate in a holding position to form together a holding element, each half-shell comprising a casing extending according to a casing axis and defining an external face and an internal face; and
a locking device able to maintain the two half-shells in the holding position around a part of the medical instrument, the locking device comprising two complementary locking elements able to cooperate with each other, one of the locking elements being arranged on one of the half-shells and the other locking element being arranged on the other half-shell;
wherein
one of the locking elements comprises a first component of a first magnet, the first component being of a first polarity,
the other of the locking elements comprising a second component of the first magnet, the second component being of a second polarity opposite to the first polarity.

2. The device according to claim 1, wherein the internal face of each half-shell defines a housing,
in the holding position, the two housings together forming a receiving space for the part of the medical instrument.

3. The device according to claim 1, further comprising a seal configured to be arranged between the part of the medical instrument and the holding element.

4. The device according to claim 2, further comprising a seal configured to be arranged between the part of the medical instrument and the holding element.

5. The device according to claim 4, wherein the seal is configured to extend throughout the entire receiving space formed by the two housings in the holding position.

6. The device according to claim 1, wherein one of the locking elements also comprises a first component of a second magnet, the first component being of a first polarity, the other of the locking elements also comprising a second component of the second magnet, the second component being of a second polarity.

7. The device according to claim 1, wherein the first magnet is a neodymium super-magnet.

8. The device according to claim 6, wherein the second magnet is a neodymium super-magnet.

9. The device according to claim 1, wherein the internal face of each half-shell defines at least one cavity filled with a filling agent.

10. The device according to claim 9, wherein each element of the first magnet is arranged in one of the cavities of the corresponding half-shell.

11. The device according to claim 6, wherein the internal face of each half-shell defines at least one cavity filled with a filling agent.

12. The device according to claim 11, wherein each element of the first magnet and each element of the second magnet is arranged in one of the cavities of the corresponding half-shell.

13. The device according to claim 1, comprising an identification element engraved on the external face of one of the half-shells.

14. An assembly comprising:
a device according to claim 1, and
a medical instrument comprising a part able to be held in the holding element formed by the two half-shells.
